(19) United States
(12) Patent Application Publication
(10) Pub. No.: US 2022/0073908 A1
(43) Pub. Date: Mar. 10, 2022

(54) METHODS OF PRODUCING HIGH DIVERSITY PEPTIDE LIBRARIES AND PROMOTING PROTEIN FOLDING

(71) Applicant: REPERTOIRE IMMUNE MEDICINES, INC., Cambridge, MA (US)

(72) Inventors: Joanna Feltham Swain, Concord, MA (US); Ellen Lovisa Larsdotter Afzelius, Belmont, MA (US); Ohad Yosefson, Concord, MA (US); Benjamin Peter Roscoe, Somerville, MA (US); Peter Lyubomirov Rogov, Winchester, MA (US); Harsh Jayeshkumar Vaidya, Somerville, MA (US); George Robert Mabry, Needham, MA (US)

(21) Appl. No.: 17/419,099

(22) PCT Filed: Jan. 3, 2020
(86) PCT No.: PCT/US2020/012231

§ 371 Date: Jun. 28, 2021

Related U.S. Application Data
(60) Provisional application No. 62/788,673, filed on Jan. 4, 2019.

(51) Int. Cl. C12N 15/10

(52) U.S. Cl. CPC C12N 15/1075

(57) ABSTRACT

The disclosure provides a peptide library with increased peptide diversity. The increase in peptide diversity can occur via cleavage of particular amino acids within a peptide. The disclosure further provides a method for promoting folding of a peptide into an active conformation.

### CROSS REFERENCE

This application claims the benefit of U.S. Provisional Application No. 62/788,673, filed Jan. 4, 2019, which is incorporated herein by reference in its entirety.

### BACKGROUND

In vitro protein production allows expression and manufacturing of small amounts of functional proteins for research and therapeutic purposes.

### INCORPORATION BY REFERENCE

Each patent, publication, and non-patent literature cited in the application is hereby incorporated by reference in its entirety as if each was incorporated by reference individually.

### SUMMARY

The present disclosure provides library compositions, methods of making libraries, and methods of promoting peptide folding.

In some embodiments, the present disclosure provides a library comprising a plurality of nucleic acid constructs encoding a plurality of peptides, wherein a nucleic acid construct of the plurality of nucleic acid constructs comprises: a) a first nucleotide sequence encoding a peptide selected from the plurality of peptides; and b) a second nucleotide sequence encoding a cleavable moiety, wherein the cleavable moiety is situated such that at least one N-terminus amino acid residue of the peptide selected from the plurality of peptides is before or within the cleavable moiety; wherein the plurality of peptides comprises greater than 1000 peptide diversity when the cleavable moiety is cleaved using an endoprotease specific to the cleavable moiety, thereby cleaving the initial amino acid residue of the peptide.

In some embodiments, the present disclosure provides a library comprising a plurality of peptides, wherein a peptide of the plurality of peptides comprises: a) at least one N-terminus amino acid residue of the peptide; b) a cleavable moiety; and c) a remainder of the peptide, wherein the at least one N-terminus amino acid residue of the peptide is before or within the cleavable moiety; wherein the plurality of peptides comprises greater than 1000 peptide diversity when the cleavable moiety is cleaved using an endoprotease specific to the cleaveable moiety, thereby cleaving the at least one N-terminus amino acid residue of the peptide.

In some embodiments, the present disclosure provides a method of making a peptide library, the method comprising: a) providing a plurality of nucleic acid constructs encoding a plurality of peptides, wherein a nucleic acid construct of the plurality of nucleic acid constructs comprises: i) a first nucleotide sequence encoding a peptide from the plurality of peptides; and ii) a second nucleotide sequence encoding a cleavable moiety, wherein the cleavable moiety is situated such that at least one N-terminus amino acid residue of the peptide selected from the plurality of peptides is before or within the cleavable moiety; b) transcribing and translating, or translating, the plurality of nucleic acid constructs; and c) cleaving the cleavable moiety using an endoprotease, optionally, simultaneously as (b), thereby cleaving the at least one N-terminus amino acid residue of the peptide from the remainder of the peptide, wherein cleavage of the at least one N-terminus amino acid residue from the peptide results in a properly folded peptide of the peptide library.

In some embodiments, the present disclosure provides a DNA construct for expression of a protein epitope, the DNA construct comprising: a) a first nucleotide sequence encoding the protein epitope; and b) a second nucleotide sequence encoding a cleavable moiety at the N-terminus of the protein epitope, wherein the cleavable moiety is situated such that at least one N-terminus amino acid residue of the protein epitope is before or within the cleavable moiety,

wherein upon transcription and translation of the DNA construct, the cleavable moiety is cleaved using an endoprotease specific to the cleavable moiety, thereby cleaving the at least one N-terminus amino acid residue of the protein epitope, and wherein the protein epitope is part of a peptide library.

In some embodiments, the present disclosure provides a RNA construct for expression of a protein epitope, the RNA construct comprising: a) a first nucleotide sequence encoding the protein epitope; and b) a second nucleotide sequence encoding a cleavable moiety at the N-terminus of the protein epitope, wherein the cleavable moiety is situated such that at least one N-terminus amino acid residue of the protein epitope is before or within the cleavable moiety, wherein upon translation of the RNA construct, the cleavable moiety is cleaved using an endoprotease specific to the cleavable moiety, thereby cleaving the at least one N-terminus amino acid residue of the protein epitope, and wherein the protein epitope is part of a peptide library.

In some embodiments, the present disclosure provides a method of folding a peptide, the method comprising: a) providing a nucleic acid construct encoding the peptide, the nucleic acid construct comprising: i) a first nucleotide sequence encoding the peptide; and ii) a second nucleotide sequence encoding a cleavable moiety, wherein the cleavable moiety is situated such that at least one N-terminus amino acid residue of the peptide is before or within the cleavable moiety; b) transcribing and translating, or translating, the nucleic acid construct; and c) cleaving the cleavable moiety using an endoprotease, optionally, simultaneously as (b), thereby cleaving the at least one N-terminus amino acid residue of the peptide from the remainder of the peptide, wherein cleavage of the at least one N-terminus amino acid residue of the peptide results in a folded peptide.

In some embodiments, the present disclosure provides a method for making a library of conformational protein epitopes, the method comprising: a) obtaining a plurality of protein epitopes encoded in a plurality of nucleic acid constructs, wherein a nucleic acid construct of the plurality further encodes a cleavable moiety at the N-terminus of a protein epitope, wherein the cleavage moiety is situated such that an initial amino acid residue of the protein epitope is before or within the cleavable moiety; b) optionally transcribing the plurality of nucleic acid constructs, wherein a plurality of ribonucleic acid molecules is transcribed from the plurality of nucleic acid constructs; c) translating the plurality of ribonucleic acid molecules, wherein the plurality of protein epitopes are translated from the plurality of ribonucleic acid molecules; and d) cleaving the cleavable moiety using a protease, optionally, simultaneously as (c), thereby cleaving the initial amino acid residue of the candidate protein epitopes from the remainder of the candidate protein epitopes.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 indicates that generated peptide in EXAMPLE 1 was digested to remove the cleavage domain.

FIG. 2 demonstrates cleavage of the SUMO domain from peptides prepared using a method described herein.

FIG. 3 provides quantification of the correct folding of peptides prepared using a method described herein.

FIG. 4 provides flow cytometry analysis of peptides prepared using a method described herein.

### DETAILED DESCRIPTION

### Definitions

As used herein, the term “cleavable moiety” refers to a motif or sequence that is cleavable. In some embodiments, the cleavage moiety comprises a protein, e.g., enzymatic, cleavage site. In some embodiments, the cleavage moiety comprises a chemical cleavage site, e.g., through exposure to oxidation/reduction conditions, light/sound, temperature, pH, pressure, etc.

As used herein, the term “endoprotease” refers to a protease that cleaves a peptide bond of a non-terminal amino acid.

As used herein, the term “high diversity” refers to having a high degree of variety.

As used herein, the term “library peptide” refers to a single peptide in the library.

As used herein, the term “N-terminus amino acid residue” refers to one or more amino acids at the N-terminus of a polypeptide.

As used herein, the term “peptide diversity” refers to a variation or variability between two or more peptides.

As used herein, the term “peptide library” refers to a plurality of peptides. In some embodiments, the library comprises one or more peptides with unique sequences. In some embodiments, each peptide in the library has a different sequence. In some embodiments, the library comprises a mixture of peptides with the same and different sequences.

As used herein, the term “high diversity peptide library” refers to a peptide library with a high degree of peptide variety. For example, a high diversity peptide library comprises about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, about 10²⁰, or more different peptides.

As used herein, the term “protein epitope” refers to a peptide sequence or structure that is predicted to interact with a partner.

As used herein, the term “protein folding” refers to spatial organization of a peptide. In some embodiments, the amino acid sequence influences the spatial organization or folding of the peptide. In some embodiments, a peptide may be folded in a functional conformation. In some embodiments, a folded peptide has one or more biological functions. In some embodiments, a folded peptide acquires a three-dimensional structure.

As used herein, the terms “small ubiquitin-like modifier moiety” or “SUMO domain” or “SUMO moiety” are used interchangeably and refer to a specific protease recognition moiety.

The present disclosure provides, for example, methods for in vitro protein production. Generally, in vitro protein production does not require gene transfection, cell culture, or extensive protein purification, but can result in a low diversity of protein expression. Conversely, mammalian based expression systems allow for increased diversity of protein expression compared to in vitro methods, but mammalian based expression systems are slow and laborious. Thus, the present disclosure provides a method for in vitro protein expression for high throughput and high diversity peptide production for use in, for example, a peptide library.

Additionally, the present disclosure provides methods for increasing proper protein folding upon generation of a protein using an in vitro protein production method described herein. In some cases, the initial methionine residue, N-formylmethionine (fMet), in in vitro bacterial systems hinders proper peptide folding and impacts peptide function. The in vitro method disclosed herein can be used to cleave the initial methionine residue after translation, which allows proper protein folding.

As used herein, the abbreviations for the 1-enantiomeric and d-enantiomeric amino acids are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). In some embodiments, the amino acid is a L-enantiomer. In some embodiments, the amino acid is a D-enantiomer.

### In Vitro Transcription/Translation.

The methods of the present disclosure comprise a method for performing in vitro transcription/translation (IVTT). For example, the methods of the present disclosure comprise a method for performing in vitro transcription/translation (IVTT) to produce a high diversity peptide library and allow for correct folding of proteins.

IVTT can allow for protein production in a cell-free environment directly from a DNA or RNA template. IVTT can be used to create, for example, mRNA display libraries, peptides, antibodies, ribosome display, DNA display, CIS display, and desired proteins.

An IVTT method used herein can be performed using, for example, a PCR product, a linear DNA plasmid, a circular DNA plasmid, or an mRNA template with a ribosome-binding site (RBS) sequence. After the appropriate template has been isolated, transcription components can be added to the template including, for example, ribonucleotide triphosphates, and RNA polymerase. After transcription has been completed, translation components can be added, which can be found in, for example, rabbit reticulocyte lysate, or wheat germ extract. In some methods, the transcription and translation can occur during a single step, in which purified translation components found in, for example, rabbit reticulocyte lysate or wheat germ extract are added at the same time as adding the transcription components to the nucleic acid template.

### Protein Folding.

A method disclosed herein can be used to facilitate proper protein folding of a peptide produced by a disclosed IVTT method for producing a peptide library with high peptide diversity.

Cell-free protein synthesis (CFPS) of a peptide can allow for the production of a peptide. Obtaining a high yield by CFPS can require the use of bacterial systems, in which the first amino acid of the translated sequence is N-formylmethionine (fMet). fMet differs from methionine by containing a neutral formyl group (HCO) instead of a positively charged amino-terminus (NH₃⁺). Although bacteria can use endogenous aminopeptidases to cleave the fMet, the removal of fMet can be either incomplete or abolished, depending on the identity of the second amino acid in the sequence. For example, the action of methionine aminopeptidase can be inefficient between fMet and asparagine.

One example of a peptide produced by CFPS is a CMV-derived peptide comprising the amino acid sequence fMet-NLVPMVATV (SEQ ID NO.: 1). The improper protein folding of this peptide can affect the ability of the protein to bind to the cognate T-cell receptor because of retention of the fMET residue. This result can occur if the protein is produced in a bacterial CFPS system that is made from a crude cell extract. Moreover, in a peptide library context, a single individual template could result in peptides with or without the fMet, or a mixture of both if the processing is merely inefficient.

However, in a reconstituted CFPS system that is composed of only purified components and completely lacks methionine aminopeptidases, all library variants can start with an fMet residue, and then this residue could be cleaved uniformly as described in the methods herein. Thus, using the IVTT methods described herein, the removal of the initial methionine amino acid allowed for successful peptide folding.

More specifically, a method as described herein can use cell-free synthesis and followed by or with a simultaneous cleavage step. The cell-free peptide synthesis can occur via use of an IVTT system. The peptide can be synthesized using the IVTT system that can both transcribe, for example, a DNA construct into RNA, and then translate the RNA into a protein. In this cell-free system for synthesis of the peptide, a nucleotide sequence can encode a methionine residue present at the N-terminus of the peptide and a cleavable moiety. The cleavable moiety can be situated such that at least one N-terminus amino acid residue of the peptide is before or within the cleavable moiety. In some embodiments, the method comprises encoding a cleavable moiety that is situated such that one N-terminus amino acid residue of the peptide is before or within the cleavable moiety. In some embodiments, the one N-terminus amino acid residue is a methionine residue. The cleavable moiety can be cleaved using a protease specific to the cleavable moiety, which can also cleave off the cleavable moiety from the remainder of the peptide.

The cleavage of the cleavable moiety can occur via the use of, for example, an amino-peptidase. In some embodiments, the cleavage of the amino acid residue occurs via the use of a methionine amino-peptidase. The methionine amino-peptidase can cleave a methionine from a peptide when the amino acid residue at position two is, for example, glycine, alanine, serine, cysteine, or proline.

An example of a cleavable moiety that can be encoded in a DNA or RNA construct as described herein includes any cleavable moiety cleaved by a protease. In some embodiments, the cleavable moiety can be a small ubiquitin-like modifier (SUMO) protein. The SUMO domain can be cleaved off of the peptide using a protease specific to SUMO. In some embodiments, the cleavable moiety can be an enterokinase cleavage site: Asp-Asp-Asp-Asp-Lys (SEQ ID NO.: 2). The protease can be, for example, Ulp1 protease or enterokinase. The Ulp1 protease can cleave off SUMO in a specific manner by recognizing the tertiary structure of SUMO, rather than an amino acid sequence. Enterokinase (enteropeptidase) can also be used to cleave after lysine at the following cleavage site: Asp-Asp-Asp-Asp-Lys (SEQ ID NO.: 2). Enterokinase can also cleave at other basic residues, depending on the sequence of the protein substrate.

During or after translation of the construct encoding the peptide, the N-terminus amino acid residue can be efficiently cleaved to produce the properly folded peptide. In some embodiments, at least one N-terminus amino acid residue is cleaved to produce the peptide. In some embodiments, one, two, three, four, five six, seven, eight, nine, ten or more N-terminus amino acid residues are cleaved to produce the peptide. The N-terminus amino acid can be any amino acid residue. The N-terminus amino acid residue can be a methionine amino acid residue. This properly folded peptide is thus not constrained to have an N-terminus methionine, and can be part of a high diversity peptide library produce by cell-free in vitro methods.

The present disclosure provides, for example, a DNA or RNA construct that can encode a peptide that can be properly folded using the methods described herein. The peptide can be any polypeptide, protein, fusion protein, or fragment thereof. For example, a DNA or RNA construct can encode a protein epitope. A DNA or RNA construct can encode a protein multimer, e.g., dimer, trimer, tetramer, etc. In some embodiments, the multimer is a homomultimer, e.g., identical subunits, or homooligomer. In some embodiments, the multimer is a heteromultimer, e.g., different subunits.

A protein epitope as described herein can refer to a specific nucleotide sequence that encodes a peptide that is predicted to bind to a protein, e.g., a receptor. A protein, e.g., a receptor, can have any level of affinity toward the protein epitope. A protein, e.g., a receptor, can have a high affinity for the protein epitope. A protein, e.g., a receptor, can have a low affinity for the protein epitope. A protein, e.g., a receptor, can have no affinity for the protein epitope. A protein, e.g., a receptor, can or cannot be specific to the protein epitope.

As another example, the protein epitope can be synthesized using the IVTT system that can both transcribe, for example, a DNA construct into RNA, and then translate the RNA into a protein. Due to the use of a cell-free system for synthesis of the protein epitope, a nucleotide sequence can encode a methionine residue at the N-terminus of the protein epitope. The N-terminus methionine residue can be cleaved from the remainder of the protein epitope as described above. Use of the methods described herein can allow for proper folding of the proteins from these DNA constructs and/or RNA constructs.

### Peptide Library.

An IVTT system as described herein can be used producing a peptide library. Peptide libraries can be used in a range of screening assays to identify potential diagnostic or therapeutic targets or agents. Peptide libraries can be used, for example, to screen for disease-specific or organ-specific peptides, to screen for peptides with therapeutic applications, to screen for peptides with diagnostic applications, to screen for tumor-targeting peptides, to screen for antibody epitopes or antigens, to screen for T cell epitopes or antigens, to screen for antimicrobial peptides, or any combination thereof. Diverse peptide libraries of appropriate quality, therefore, have many valuable uses.

The IVTT system as described herein can be used for producing a high diversity peptide library. The IVTT system as described herein can be used for producing a high diversity peptide library via high throughput methods. The produced high diversity peptide library can comprise correctly folded peptides as described above.

Peptide diversity can be assessed based on, for example, direct measurement of the different types of peptides present in a particular library. Peptide diversity can also be measured by determining the distribution of single amino acids and dipeptides in a sample. A high diversity peptide library can be a library comprising no less than 10³ peptides that are unique compared to each other. Peptide diversity can be determined by, for example, sequencing the individual peptides in the library, or by using mass spectrometry to measure different species in the library.

A peptide library created using the methods disclosed herein can have a peptide diversity of, for example, about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, about 10²⁰, or more. In some embodiments, a peptide library described herein has a peptide diversity of greater than or equal to about 10⁹.

A method disclosed herein can be used to create a peptide library using, for example, a cell-free method. The cell-free library synthesis can occur via use of an IVTT system. The peptide can be synthesized using the IVTT system that can both transcribe, for example, a DNA construct into RNA, and then translate the RNA into a protein. A nucleotide sequence encoding a methionine residue at the N-terminus of the peptide and a cleavable moiety can be encoded in the DNA construct or RNA construct. The cleavable moiety is situated such that at least one N-terminus amino acid residue of the peptide is before or within the cleavable moiety. In some embodiments, the method comprises encoding a cleavable moiety that is situated such that one N-terminus amino acid residue of the peptide is before or within the cleavable moiety. In some embodiments, the one N-terminus amino acid residue is a methionine residue. The cleavable moiety can be cleaved using a protease specific to the cleavable moiety, which can also cleave off the cleavable moiety from the remainder of the peptide.

The cleavage of the cleavable moiety can occur via the use of, for example, an amino-peptidase. In some embodiments, the cleavage of the amino acid residue occurs via the use of a methionine amino-peptidase. The methionine amino-peptidase can cleave a methionine from a peptide when the amino acid residue at position two is, for example, glycine, alanine, serine, or proline.

An example of a cleavable moiety that can be encoded in a DNA or RNA construct as described herein includes any cleavable moiety cleaved by a protease. In some embodiments, the cleavable moiety can be a small ubiquitin-like modifier (SUMO) protein. The SUMO domain can be cleaved off of the peptide using a protease specific to SUMO. In some embodiments, the cleavable moiety can be an enterokinase cleavage site: Asp-Asp-Asp-Asp-Lys (SEQ ID NO.: 2). The protease can be, for example, Ulp1 protease or enterokinase. The Ulp1 protease can cleave off SUMO in a specific manner by recognizing the tertiary structure of SUMO, rather than an amino acid sequence. Enterokinase (enteropeptidase) can also be used to cleave after lysine at the following cleavage site: Asp-Asp-Asp-Asp-Lys (SEQ ID NO.: 2). Enterokinase can also cleave at other basic residues, depending on the sequence of the protein substrate.

During or after translation of the construct encoding the library peptide, an N-terminus amino acid residue can be cleaved to produce the peptide for the high diversity peptide library. In some embodiments, at least one N-terminus amino acid residue is cleaved to produce the peptide. In some embodiments, one, two, three, four, five six, seven, eight, nine, ten, twenty, thirty, forty, fifty, sixty, seventy, eighty, ninety, one hundred, or more N-terminus amino acid residues are cleaved to produce the library peptide. The N-terminus amino acid can be any amino acid residue. The N-terminus amino acid residue can be a methionine amino acid residue.

A peptide library can be used to, for example, identify peptide-target protein interactions, such as identify receptor/ligand interactions, perform protein conformation studies, develop high affinity and low antibodies, identify peptide mimetics, identify immunogenic peptides, identify binding patterns between peptides (e.g., between single peptides, immunogenic peptides, or peptide mimetics), identify immune cell receptor/antigen pairs, develop vaccines, perform protein kinase studies, perform protease studies, and perform drug design studies. Thus, having a diverse array of peptides in a peptide library can be useful in ensuring that accurate targets are identified, and that resulting biological assays are performed to yield accurate results.

In some embodiments, a method disclosed herein can be used to increase the peptide diversity of a peptide library to identify protein-protein interactions. The protein epitopes can bind to, for example, receptors, antibodies, immune cell receptors (BCRs, MHCs, TCRs), cell surface protein, kinases, proteases, drugs, or others.

The present disclosure provides, for example, a DNA or RNA construct that can encode a library peptide. The library peptide can be any polypeptide, protein, protein fusion, or fragment thereof. For example, a DNA or RNA construct can encode a library peptide comprising a protein epitope. A DNA or RNA construct can encode a protein multimer, e.g., dimer, trimer, tetramer, etc. that comprises the library peptide. In some embodiments, the multimer is a homomultimer, e.g., identical subunits, or homooligomer. In some embodiments, the multimer is a heteromultimer, e.g., different subunits.

A library peptide comprising a protein epitope as described herein can refer to a specific nucleotide sequence that encodes a library peptide that is predicted to bind to a particular protein, e.g., a receptor. A protein, e.g., a receptor, can have any level of affinity toward the protein epitope. A protein, e.g., a receptor, can have a high affinity for the protein epitope. A protein, e.g., a receptor, can have a low affinity for the protein epitope. A protein, e.g., a receptor, can have no affinity for the protein epitope. A protein, e.g., a receptor, can or cannot be specific to the protein epitope.

As another example, the library peptide comprising a protein epitope can be synthesized using the IVTT system that can both transcribe, for example, a DNA construct into RNA, and then translate the RNA into a protein. Due to the use of a cell-free system for synthesis of the protein epitope, a nucleotide sequence can encode a methionine residue at the N-terminus of the protein epitope. The N-terminus methionine residue can be cleaved from the remainder of the protein epitope as described above.

### Methods Used to Detect Binding of Protein Epitope.

After IVTT of the DNA or RNA construct as described herein, the binding of the protein epitope to, for example, a target receptor, can be determined. The binding can be assessed using FACS. After translation of the DNA or RNA construct as described herein, the protein epitope can be exposed to, for example, a sample of target receptor-expressing cells. The cells can then be stained with a fluorescent dye that can be specific to, for example, the protein epitope. The stained cells can then be sorted using FACS based on strength of the fluorescent signal. The stain used for FACS analysis can be, for example phycoerythrin (PE), fluorescein isothiocyanate (FITC), 7-Aminoactinomycin D (7-AAD), allophycocyanin (APC), or any combination or modification of the foregoing.

After translation of the DNA or RNA constructs as described herein, the peptide library can be exposed to, for example, a sample of target receptors, e.g., a plurality of receptors, e.g., a pool of different receptors. Interacting library peptide and receptor can then be stained with a fluorescent dye that can be specific to, for example, the protein epitope. Other methods in the art for detection of protein interactions include, but are not limited to, ELISA, western blot, co-immunoprecipitation, immunoelectrophoresis, affinity purification, mass spectrometry, etc.

### Constructs.

A construct described herein can be, for example, a DNA or RNA construct. A construct can also contain artificial nucleic acids. The nucleic acids of the construct can include, for example, genomic DNA, cDNA, tRNA, mRNA, rRNA, modified RNA, miRNA, gRNA, and siRNA, or other RNAi molecule.

A construct as described herein can have a length of at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

### Linkers.

The constructs described herein can include a linker between different domains of the construct. A linker may be a chemical bond, e.g., one or more covalent bonds or non-covalent bonds. In some embodiments, the linker is a covalent bond. In some embodiments, the linker is a non-covalent bond. In some embodiments, a linker is a peptide linker. Such a linker may be between 2-30 amino acids, or longer. In some embodiments, a linker can be used, e.g., to space the hydrogel from the target molecule. In some embodiments, for example, a linker can be positioned between a target molecule and another target molecule. In some embodiments, a linker can be positioned between domains in the target molecule, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release the target molecule from the hydrogel, but sufficiently stable to prevent premature cleavage.

The most commonly used flexible linkers have sequences consisting primarily of stretches of Gly and Ser residues (“GS” linker). Flexible linkers can be useful for joining domains that require a certain degree of movement or interaction and can include small, non-polar (e.g. Gly) or polar (e.g. Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties.

Rigid linkers can be used to keep a fixed distance between domains of the construct and to maintain the independent functions of the respective domains. Rigid linkers can have, for example, an alpha helix-structure or Pro-rich sequence, (XP)n, with X designating any amino acid.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of linkers include a hydrophilic or hydrophobic linkers, such as a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more components of a promoting agent (e.g. two polypeptides). Non-covalent linkers are also included, such as hydrophobic lipid globules to which the target molecule is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine or other hydrophobic residue. Components of target molecule or hydrogel may be linked using charge-based chemistry, such that a positively charged component of the target molecule or hydrogel is linked to a negative charge of another molecule.

### Peptides or Proteins.

The present disclosure provides, for example, a DNA or RNA construct that can encode a peptide. The peptide can be any polypeptide, protein, or fragment thereof.

The peptide, once translated, can have a length from about 3 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful. In some embodiments, the peptide can have a length of about 5 to about 200 amino acids, about 15 to about 150 amino acids, about 20 to about 125 amino acids, about 25 to about 100 amino acids, or any range therein. The protein epitope, once translated, can have a length from about 5 to about 200 amino acids, about 15 to about 150 amino acids, about 20 to about 125 amino acids, about 25 to about 100 amino acids, or any range therein.

A peptide library described herein can contain an array platform that contains a plurality of individual features on the surface of the array. Each feature can contain a plurality of individual peptides synthesized in situ or in vitro on the surface of the array or spotted on the surface, wherein the molecules are identical within a feature, but the sequence or identity of the molecules differ between features. Such array molecules include the synthesis of large synthetic peptide arrays.

The peptide arrays can include control sequences that are known to bind to, for example, cell receptors. Binding patterns to control sequences and to library peptides can be measured to qualify the arrays and the assay process.

In some embodiments, the peptide library comprises about 100, about 500, about 1000, about 2000, about 3000, about 4000, about 5,000, about 6000, about 7000, about 8000, about 9000, about 10,000, about 15,000, about 20,000, about 30,000, about 40,000, about 50,000, about 100,000, about 200,000, about 300,000, about 400,000, about 500,000, or more peptides having different sequences.

Platforms herein can also include peptides in microtiter plates for determining protein interactions of the protein epitopes provided herein. In some embodiments, microtiter plates include but are not limited to 96 well, 384 well, 1536 well, 3456 well, and 9600 well plates. In some embodiments, more than one peptide is present in each well of a microtiter plate, i.e., the peptides are pooled and individual peptides interacting with a target protein are determined by deconvolution of the positive and negative wells in the assay.

### EXAMPLES

The following examples are included to further describe some aspects of the present disclosure, and should not be used to limit the scope of the invention.

### Example 1: In Vitro Translation of a Peptide Library

This Example demonstrates cell-free synthesis (CFPS) of a protein.

CFPS of a peptide library enables the production of a broad range of various peptides. Obtaining a high yield by CFPS requires the use of bacterial systems, in which the first amino acid of the translated sequence is N-formylmethionine (fMet). This residue differs from methionine by containing a neutral formyl group (HCO) instead of a positively charged amino-terminus (NH₃⁺). Although bacteria are utilizing endogenous aminopeptidases to cleave the fMet, the removal of fMet could be either incomplete or abolished, depending on the identity of the second amino acid in the sequence. For example, methionine aminopeptidase excises inefficiently between fMet and asparagine. Consequently, a CMV derived peptide, a model peptide in this system, will ultimately be produced as fMet-NLVPMVATV (SEQ ID NO.: 1) in a single-chain HLA or MHC design; thus, the entire molecule will not fold correctly and will not recognize its cognate T-cell receptor. This result is anticipated if the protein will be produced in bacterial CFPS system that is made from a crude cell extract. Moreover, in a library context a single individual template could result in peptides with the fMet, or without it, or a mixture of both if the processing is merely inefficient. In a reconstituted CFPS system that is composed of only purified components and completely lacks methionine aminopeptidases, all library variants will start with an fMet residue.

To solve this problem, constructs were engineered to include genes encoding an enzymatic cleavage domain and the library peptide. Removal of at least the initial methionine amino acid allowed the upper limit of the peptide library to include greater diversity, e.g., 20^{x}, where x is the length of the peptide, while inclusion of the methionine residue would restrict the library diversity to 20^{(x-1)}. Furthermore, removal of the initial methionine amino acid allowed for successful peptide folding and expression of the peptides at measurable levels.

Peptides were synthesized under cell-free conditions. All CFPS components were thawed and mixed on ice and then moved to the relevant temperature to initiate the reaction. To one tube, the following reagents were added: 40% (v/v) PURExpress solution A, 30% (v/v) of PURExpress solution B (E6800L, New England Biolabs, Inc.), 0.8 U/μ1 reaction of RNase inhibitor (10777019, ThermoFischer Scientific), 4% (v/v) of each disulfide enhancers 1 and 2 (E6820L, New England Biolabs, Inc.), 0.004 U/μ1 reaction of SUMO-protease (chosen for its complete removal of excision overhangs (scar-less) after cleavage) diluted in PBS (12588018, Invitrogen), nuclease-free water, and 20 ng/μ1 reaction of the corresponding plasmid DNA encoding the desired CFPS product. Four different temperatures of CFPS were tested: 20, 25, 30 and 37° C. In each indicated time point, samples were taken and the reactions were stopped by placing the tubes on ice and adding EDTA to a final concentration of 2 mM.

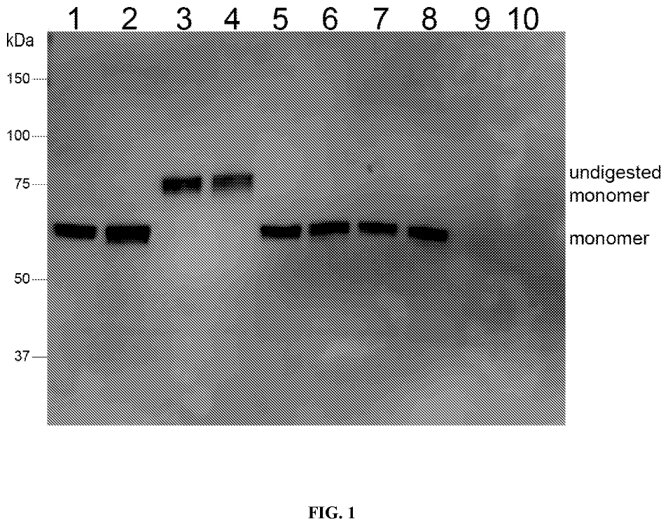

FIG. 1 indicates that peptide was digested to remove the cleavage domain to increase diversity. Lane pairs 1-2, 3-4, 5-6, 7-8 and 9-10 represent CFPS reactions performed on a template without a SUMO domain, a template with a SUMO domain where no protease was added, a template with a SUMO domain in which the protease was added after the reaction was completed, a template with a SUMO domain in which the protease was present during the reaction and a reaction that lacked a DNA template, respectively. Samples in odd numbered lanes were prepared for gel electrophoresis under reduced conditions with the addition of 100 mM DTT. All reactions were terminated by placing the tubes on ice after 4 hr at room temperature. 4 U/reaction of SUMO protease were added to samples 3-8. In the reactions loaded in lanes 5-6, the protease was added after the tube was placed on ice together with 10 mM EDTA and then transferred to room temperature for additional 3.5 hours before placing on ice again.

Separate constructs with an enterokinase cleavage domain prior to the peptide also showed protein production and cleavage when assessed by western blot.

Western blotting was performed to determine total protein yield. Each CFPS sample was mixed with water, 4× sample buffer and 1M DTT, boiled at 95° C. for 5 minutes, and then loaded on a 10% SDS-PAGE gel. Samples were blotted using an HRP-anti-FLAG antibody.

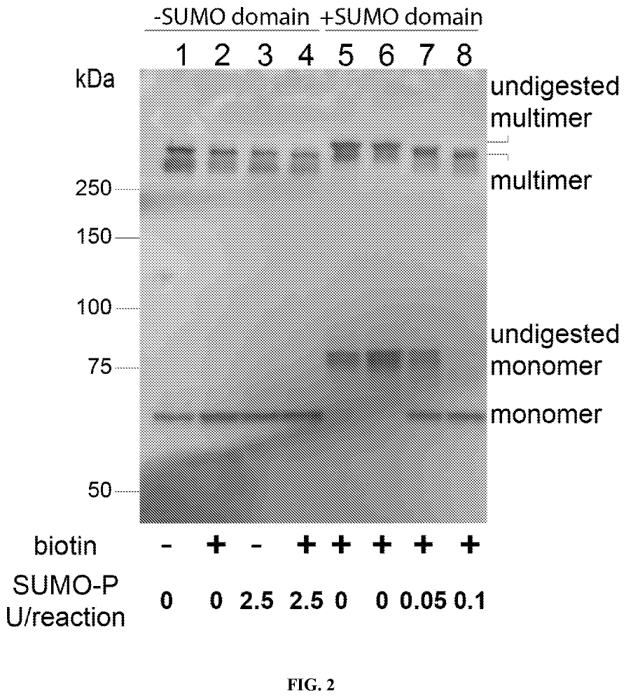

FIG. 2 show samples from the CFPS reaction that contained templates with or without the SUMO domain. Reactions were terminated by placing the tubes on ice after 4 hr at room temperature. Samples were prepared for western blotting as above with the exclusion of the 95° C. boiling step. These results demonstrate that the SUMO protease cleaves the CFPS product if and only if the SUMO domain is present.

### Example 2: Assessing the 3-D Structure of an In Vitro Translated Protein

This Example demonstrates that the CFPS protein prepared in EXAMPLE 1 folded into a recognizable three-dimensional structure.

The CFPS protein was tested for conformational recognition by an antibody. Proteins that are misfolded or unfolded are not recognized by the antibody. The CFPS protein with the cleaved enzymatic domain was folded and recognized by the conformation-specific antibody.

Protein expression was measured through an ELISA. Plates were coated with anti-streptavidin antibody (410501, Biolegend) diluted in 100 mM bicarbonate/carbonate coating buffer and incubated overnight at 4° C. Then, the plates were washed three times by filling the wells with washing buffer (PBS supplemented with 0.05% tween-20) and blocked for 2 hr at room temperature by filling the wells with blocking buffer (washing buffer supplemented with 2% (V/V) BSA). The wells were then filled with serial dilutions of each CFPS protein in blocking buffer followed by 1 hr incubation at room temperature. Then, the plates were washed three times with washing buffer and incubated with 0.15 μg/ml horseradish peroxidase antibodies specific to the protein diluted in blocking buffer for 1 hr at room temperature.

After three more washes, colors were developed with the addition of 3,3′,5,5′ tetramethylbenzidine substrate to each well and the reaction was stopped by adding a commercial stop solution. The absorbance at 450 nm was measured using a plate reader. The absorbance values were taken in duplicate and averaged. Plates were covered with adhesive plastic and were gently agitated on a rotator during all incubations. The concentration of each sample was interpolated from a standard curve of a positive control protein.

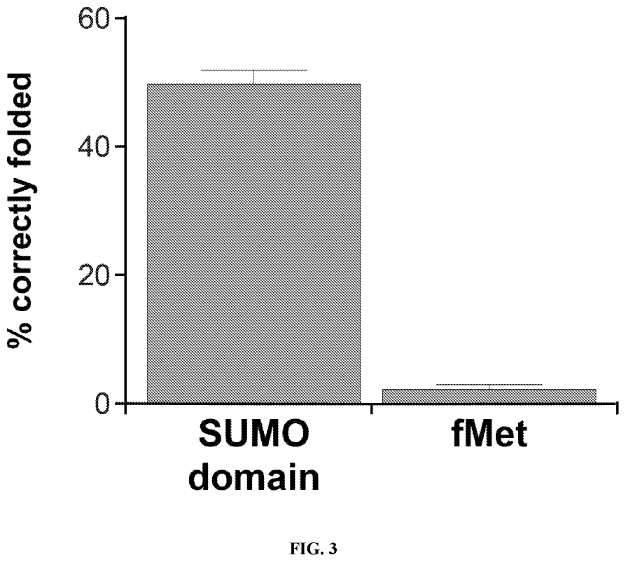

FIG. 3 shows ELISA detection of linear epitopes or a conformation epitope, from which the correctly folded percentage was calculated. The SUMO protease was added to both CFPS reactions. FIG. 3 indicates whether SUMO cleaved peptide or the fMet product demonstrated correct folding by recognition with the conformational epitope antibody.

For FACS staining, CMV enriched T-cells (Donor 153, Astarte 3835FE18, Cat #1049) were used. Wells of a 96-well round bottomed microtiter plate were filled with T-cells and the cells were washed once with ice-cold FACS buffer (D-PBS, 2 mM EDTA and 2% (V/V) fetal bovine serum), spun at 300 g at 4° C., and the supernatant was removed. Then, the relevant wells were blocked with Fc receptor blocking solution for 30 minutes at 4° C. under gentle agitation, washed with FACS buffer, and the supernatant was removed. FACS buffer was added to the compensation control wells.

In the next step, the cells were incubated for 30 minutes at 4° C. with either 20 nM positive control or dilutions of samples taken from the indicated CFPS reactions as described above, and then washed once with FACS buffer. 100 nM detection antibody diluted in FACS buffer was added to each well and the plate was incubated at 4° C. for 30 minutes in the dark, followed by two washes with PBS, and staining with fixable viability dye APC-efluor780 (1:8000 dilutions, 50 μl/well) for 15 min at room temperature. The plate was then washed twice with FACS buffer and fixed with fixation buffer PBS, 3.7% formaldehyde (v/v), 2% FBS (v/v)). Finally, the samples were transferred to FACS tubes for analysis.

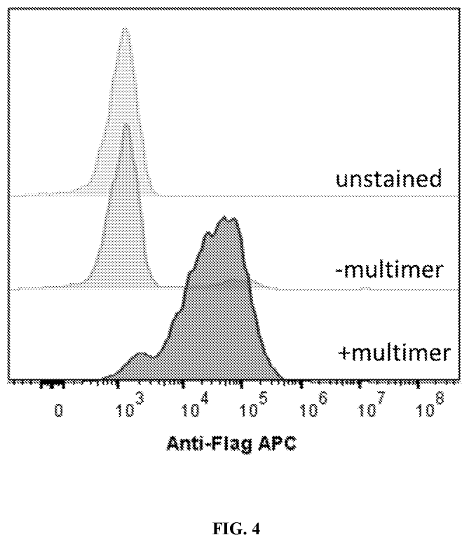

FIG. 4 shows the right shift of the multimer protein after SUMO cleavage.

What is claimed is:

1. A library comprising a plurality of nucleic acid constructs encoding a plurality of peptides, wherein a nucleic acid construct of the plurality of nucleic acid constructs comprises:
a) a first nucleotide sequence encoding a peptide selected from the plurality of peptides; and
b) a second nucleotide sequence encoding a cleavable moiety, wherein the cleavable moiety is situated such that at least one N-terminus amino acid residue of the peptide selected from the plurality of peptides is before or within the cleavable moiety;
wherein the plurality of peptides comprises greater than 1000 peptide diversity when the cleavable moiety is cleaved using an endoprotease specific to the cleavable moiety, thereby cleaving the initial amino acid residue of the peptide.

2. The library of claim 1, wherein the peptide selected from the plurality of peptides binds to a target receptor.

3. The library of claim 1, wherein the peptide selected from the plurality of peptides binds to at least one selected from an antibody, immune cell receptor (BCR, MHC, TCR), cell surface protein, kinase, protease, drug, or any combination thereof.

4. The library of any one of claims 1-3, wherein the cleavable moiety is a small ubiquitin-like modifier (SUMO) moiety.

5. The library of any one of claims 1-3, wherein the cleavable moiety is SEQ ID NO.: 2.

6. The library of any one of claims 1-5, wherein the endoprotease is enterokinase.

7. The library of any one of claims 1-6, wherein the endoprotease is Ulp1 peptidase.

8. The library of any one of claims 1-7, wherein the at least one N-terminus amino acid residue is methionine.

9. The library of any one of claims 1-8, wherein the cleavage of the cleavable moiety occurs during transcription and translation of the nucleic acid construct.

10. The library of any one of claims 1-9, wherein the cleavage of the cleavable moiety occurs after transcription and translation of the nucleic acid construct.

11. The library of any one of claims 1-10, wherein the library has a peptide diversity greater than about 10³, about 10⁴, about 10⁵, about 10⁶, about 10⁷, about 10⁸, about 10⁹, about 10¹⁰, about 10¹¹, about 10¹², about 10¹³, or about 10¹⁴ peptide diversity.

12. The library of any one of claims 1-11, wherein the library is a peptide library.

13. The library of any one of claims 1-12, wherein the nucleic acid construct is a DNA construct.

14. The library of any one of claims 1-13, wherein the nucleic acid construct is an RNA construct.

15. A library comprising a plurality of peptides, wherein a peptide of the plurality of peptides comprises:
a) at least one N-terminus amino acid residue of the peptide;
b) a cleavable moiety; and
c) a remainder of the peptide, wherein the at least one N-terminus amino acid residue of the peptide is before or within the cleavable moiety;
wherein the plurality of peptides comprises greater than 1000 peptide diversity when the cleavable moiety is cleaved using an endoprotease specific to the cleaveable moiety, thereby cleaving the at least one N-terminus amino acid residue of the peptide.

16. The library of claim 15, wherein the peptide of the plurality of peptides binds to a cell receptor.

17. The library of claim 15, wherein the peptide of the plurality of peptides binds to a T-cell receptor (TCR).

18. The library of any one of claims 15-17, wherein the cleavable moiety is SEQ ID NO.: 2.

19. The library of any one of claims 15-17, wherein the cleavable moiety is a small ubiquitin-like modifier (SUMO) moiety.

20. The library of any one of claims 15-19, wherein the endoprotease is enterokinase.

21. The library of any one of claims 15-19, wherein the endoprotease is Ulp1 peptidase.

22. The library of any one of claims 15-21, wherein the at least one N-terminus amino acid residue is methionine.

23. The library of any one of claims 15-22, wherein the peptide of the plurality of peptides is encoded by a first nucleotide sequence, wherein the first nucleotide sequence is part of a DNA construct.

24. The library of claim 23, wherein the DNA construct further comprises a second nucleotide sequence, wherein the second nucleotide sequence encodes the cleavable moiety.

25. The library of claim 24, wherein the cleavage of the cleavable moiety occurs during transcription and translation of the DNA construct.

26. The library of claim 24, wherein the cleavage of the cleavable moiety occurs after transcription and translation of the DNA construct.

27. The library of any one of claims 15-22, wherein the peptide of the plurality of peptides is encoded by a first nucleotide sequence, wherein the first nucleotide sequence is part of a RNA construct.

28. The library of claim 27, wherein the RNA construct further comprises a second nucleotide sequence, wherein the second nucleotide sequence encodes the cleavable moiety.

29. The library of any one of claims 27-28, wherein cleavage of the cleavable moiety occurs during translation of the RNA construct.

30. The library of any one of claims 28-29, wherein cleavage of the cleavable moiety occurs after translation of the RNA construct.

31. The library of any one of claims 15-30, wherein the library has a peptide diversity greater than about 10³, about 10⁴, about 10⁵, about 10⁶, about 10⁷, about 10⁸, about 10⁹, about 10¹⁰, about 10¹¹, about 10¹², about 10¹³, or about 10¹⁴ peptide diversity.

32. The library of any one of claims 15-31, wherein the library is a peptide library.

33. A method of making a peptide library, the method comprising:
a) providing a plurality of nucleic acid constructs encoding a plurality of peptides, wherein a nucleic acid construct of the plurality of nucleic acid constructs comprises:
i) a first nucleotide sequence encoding a peptide from the plurality of peptides; and
ii) a second nucleotide sequence encoding a cleavable moiety, wherein the cleavable moiety is situated such that at least one N-terminus amino acid residue of the peptide selected from the plurality of peptides is before or within the cleavable moiety;
b) transcribing and translating, or translating, the plurality of nucleic acid constructs; and
c) cleaving the cleavable moiety using an endoprotease, optionally, simultaneously as (b), thereby cleaving the at least one N-terminus amino acid residue of the peptide from the remainder of the peptide,
wherein cleavage of the at least one N-terminus amino acid residue from the peptide results in a properly folded peptide of the peptide library.

34. The method of claim 33, wherein the peptide from the plurality of peptides binds to a cell receptor.

35. The method of claim 33, wherein the peptide from the plurality of peptides binds to a T-cell receptor (TCR).

36. The method of any one of claims 33-35, wherein the cleavable moiety is a protein.

37. The method of any one of claims 33-35, wherein the cleavable moiety is SEQ ID NO.: 2.

38. The method of any one of claims 33-35, wherein the cleavable moiety is a small ubiquitin-like modifier (SUMO) moiety.

39. The method of any one of claims 33-37, wherein the endoprotease is enterokinase.

40. The method of any one of claims 33-37, wherein the endoprotease is Ulp1 peptidase.

41. The method of any one of claims 33-40, wherein the at least one N-terminus amino acid residue is methionine.

42. The method of any one of claims 33-41, wherein the library has a peptide diversity greater than about 10³, about 10⁴, about 10⁵, about 10⁶, about 10⁷, about 10⁸, about 10⁹, about 10¹⁰, about 10¹¹, about 10¹², about 10¹³, or about 10¹⁴ peptide diversity.

43. The method of any one of claims 33-42, wherein the nucleic acid construct is a DNA construct.

44. The method of any one of claims 33-42, wherein the nucleic acid construct is an RNA construct.

45. A DNA construct for expression of a protein epitope, the DNA construct comprising:
a) a first nucleotide sequence encoding the protein epitope; and
b) a second nucleotide sequence encoding a cleavable moiety at the N-terminus of the protein epitope, wherein the cleavable moiety is situated such that at least one N-terminus amino acid residue of the protein epitope is before or within the cleavable moiety, wherein upon transcription and translation of the DNA construct, the cleavable moiety is cleaved using an endoprotease specific to the cleavable moiety, thereby cleaving the at least one N-terminus amino acid residue of the protein epitope, and
wherein the protein epitope is part of a peptide library.

46. The DNA construct of claim 45, wherein the candidate peptide binds to a cell receptor.

47. The DNA construct of claim 45, wherein the candidate peptide binds to a T-cell receptor (TCR).

48. The DNA construct of any one of claims 45-47, wherein the cleavable moiety is a small ubiquitin-like modifier (SUMO) moiety.

49. The DNA construct of any one of claims 45-47, wherein the cleavable moiety is SEQ ID NO.: 2.

50. The DNA construct of any one of claims 45-49, wherein the endoprotease is enterokinase.

51. The DNA construct of any one of claims 45-49, wherein the endoprotease is Ulp1 peptidase.

52. The DNA construct of any one of claims 45-51, wherein the at least one N-terminus acid residue is methionine.

53. The DNA construct of any one of claims 45-52, wherein the cleavage of the cleavable moiety occurs during transcription and translation of the DNA construct.

54. The DNA construct of any one of claims 45-52, wherein the cleavage of the cleavable moiety occurs after transcription and translation of the DNA construct.

55. The DNA construct of any one of claims 45-54, wherein the peptide library has a peptide diversity greater than about 10³, about 10⁴, about 10⁵, about 10⁶, about 10⁷, about 10⁸, about 10⁹, about 10¹⁰, about 10¹¹, about 10¹², about 10¹³, or about 10¹⁴ peptide diversity.

56. A RNA construct for expression of a protein epitope, the RNA construct comprising:
a) a first nucleotide sequence encoding the protein epitope; and
b) a second nucleotide sequence encoding a cleavable moiety at the N-terminus of the protein epitope, wherein the cleavable moiety is situated such that at least one N-terminus amino acid residue of the protein epitope is before or within the cleavable moiety, wherein upon translation of the RNA construct, the cleavable moiety is cleaved using an endoprotease specific to the cleavable moiety, thereby cleaving the at least one N-terminus amino acid residue of the protein epitope, and
wherein the protein epitope is part of a peptide library.

57. The RNA construct of claim 56, wherein the candidate protein binds to a cell receptor.

58. The RNA construct of claim 56, wherein the candidate protein binds to a T-cell receptor (TCR).

59. The RNA construct of any one of claims 56-58, wherein the cleavable moiety is a protein.

60. The RNA construct of any one of claims 56-58, wherein the cleavable moiety is a small ubiquitin-like modifier (SUMO) moiety.

61. The RNA construct of any one of claims 56-58, wherein the cleavable moiety is SEQ ID NO.: 2.

62. The RNA construct of any one of claims 56-60, wherein the endoprotease is enterokinase.

63. The RNA construct of any one of claims 56-60, wherein the endoprotease is Ulp1 peptidase.

64. The RNA construct of any one of claims 56-63, wherein the at least one N-terminus amino acid residue is methionine.

65. The RNA construct of any one of claims 56-64, wherein the cleavage of the cleavable moiety occurs during translation of the RNA construct.

66. The RNA construct of any one of claims 56-64, wherein the cleavage of the cleavable moiety occurs after translation of the RNA construct.

67. The RNA construct of any one of claims 56-66, wherein the peptide library has a peptide diversity greater than about 10³, about 10⁴, about 10⁵, about 10⁶, about 10⁷, about 10⁸, about 10⁹, about 10¹⁰, about 10¹¹, about 10¹², about 10¹³, or about 10¹⁴ peptide diversity.

68. A method of folding a peptide, the method comprising:
a) providing a nucleic acid construct encoding the peptide, the nucleic acid construct comprising:
i) a first nucleotide sequence encoding the peptide; and
ii) a second nucleotide sequence encoding a cleavable moiety, wherein the cleavable moiety is situated such that at least one N-terminus amino acid residue of the peptide is before or within the cleavable moiety;
b) transcribing and translating, or translating, the nucleic acid construct; and
c) cleaving the cleavable moiety using an endoprotease, optionally, simultaneously as (b), thereby cleaving the at least one N-terminus amino acid residue of the peptide from the remainder of the peptide,
wherein cleavage of the at least one N-terminus amino acid residue of the peptide results in a folded peptide.

69. The method of claim 68, wherein the peptide binds to a cell receptor.

70. The method of claim 68, wherein the peptide binds to a T-cell receptor (TCR).

71. The method of any one of claims 68-70, wherein the cleavable moiety is a small ubiquitin-like modifier (SUMO) moiety.

72. The method of any one of claims 68-70, wherein the cleavable moiety is SEQ ID NO.: 2.

73. The method of any one of claims 68-72, wherein the endoprotease is enterokinase.

74. The method of any one of claims 68-72, wherein the endoprotease is Ulp1 peptidase.

75. The method of any one of claims 68-73, wherein the at least N-terminal residue is methionine.

76. The method of any one of claims 68-75, wherein the nucleic acid construct is a DNA construct.

77. The method of any one of claims 68-75, wherein the nucleic acid construct is an RNA construct.

78. A method for making a library of conformational protein epitopes, the method comprising:
a) obtaining a plurality of protein epitopes encoded in a plurality of nucleic acid constructs, wherein a nucleic acid construct of the plurality further encodes a cleavable moiety at the N-terminus of a protein epitope, wherein the cleavage moiety is situated such that an initial amino acid residue of the protein epitope is before or within the cleavable moiety;
b) optionally transcribing the plurality of nucleic acid constructs, wherein a plurality of ribonucleic acid molecules is transcribed from the plurality of nucleic acid constructs;
c) translating the plurality of ribonucleic acid molecules, wherein the plurality of protein epitopes are translated from the plurality of ribonucleic acid molecules; and
d) cleaving the cleavable moiety using a protease, optionally, simultaneously as (c), thereby cleaving the initial amino acid residue of the candidate protein epitopes from the remainder of the candidate protein epitopes.

79. The method of claim 78, wherein the cleavable moiety is a small ubiquitin-related modifier (SUMO) moiety.

80. The method of claim 78, wherein the cleavable moiety is SEQ ID NO.: 2.


SEQUENCE LISTING (3 sequences)

SEQ ID NO: 1 - protein, 10 aa, Artificial Sequence
  Description of Artificial Sequence Synthetic peptide
  MOD_RES (1)..(1): N-formylmethionine
MNLVPMVATV

SEQ ID NO: 2 - protein, 5 aa, Artificial Sequence
  Description of Artificial Sequence Synthetic peptide
DDDDK

SEQ ID NO: 3 - protein, 5 aa, Artificial Sequence
  Description of Artificial Sequence Synthetic peptide
CPRSC